(19) United States
(12) Patent Application Publication
(10) Pub. No.: US 2025/0381710 A1
(43) Pub. Date: Dec. 18, 2025

(54) METHOD AND DEVICE FOR REPROCESSING OF COMPOSITE PLASTIC PARTS

(71) Applicant: BLANCO GMBH + CO KG, Oberderdingen (DE)

(72) Inventor: Andreas HAJEK, Weingarten (DE)

(73) Assignee: BLANCO GMBH + CO KG, Oberderdingen (DE)

(21) Appl. No.: 19/239,014

(22) Filed: Jun. 16, 2025

(30) Foreign Application Priority Data
Jun. 18, 2024 (DE) 10 2024 205 621.9

(51) Int. Cl. B29B 17/02; B29B 17/04; B29K 33/00; B29K 105/26; B29K 503/04

(52) U.S. Cl. CPC B29B 17/02; B29B 2017/0231; B29B 2017/0468; B29B 2017/0496; B29K 2033/12; B29K 2105/26; B29K 2503/04

(57) ABSTRACT

A process for reprocessing composite plastic parts, for example molded parts produced from curable casting compounds, such as sanitary basins, comprising at least one filler and at least one polymer, in particular polymethyl methacrylate is provided.

The invention relates to a method for reprocessing composite plastic parts, for example molded parts produced from curable casting compounds, such as sanitary basins, comprising at least one filler and at least one polymer, in particular polymethyl methacrylate.

The invention also relates to a device for reprocessing composite plastic parts.

The importance of recycling composite plastics is growing due to the increasing amount of plastic waste and the increasing efforts to conserve resources and reduce environmental pollution. Recycling should make it possible to reuse materials and reduce the need for new raw materials, which should bring both ecological and economic benefits.

A process for recycling polymethyl methacrylate is known from WO 00/17149 A1, wherein the polymer material is brought into contact with hot mechanically fluidized solids as fluidized material in a reactor and depolymerized in the process. The resulting vapors are discharged and condensed. The resulting pyrolysis oil contains methyl methacrylate, which can be used to produce new polymers. As part of the process, the hot fluidized bed is continuously fed in at one end of the reactor and discharged at the other end.

However, when using this process to recycle composite plastics, which usually contain a filler such as carbon fibers or inorganic material in addition to polymers, the disadvantage is that the separated filler is discharged from the reactor together with the fluidized material and therefore has to be separated from the fluidized material in an additional process step.

It is therefore a goal of the present invention to provide a method and a device for reprocessing composite plastic parts, which enable simple and efficient reprocessing of composite plastic parts.

A further goal of the present invention is to provide an alternative method and an alternative device for reprocessing composite plastic parts.

In one embodiment, the present invention solves the above-mentioned goals with a process for recycling composite plastic parts, for example molded parts produced from curable casting compounds, such as sanitary basins, comprising at least one filler and at least one polymer, in particular polymethyl methacrylate, with the steps:
crushing at least one composite plastic part into a feed material,
feeding the feed material into a fluidized bed reactor with at least one fluidized bed,
depolymerizing the at least one polymer of the feed material by pyrolysis to form a pyrolysis product, in particular comprising methyl methacrylate,
separating the filler from the pyrolysis product, wherein the separated filler and the fluidized material are essentially similar, in particular identical, in terms of material and/or size distribution,
discharging a fluid stream comprising the pyrolysis product from the fluidized bed reactor, and
discharging at least part of the separated filler together with at least part of the fluidized bed material from the fluidized bed reactor.

In one embodiment, the present invention achieves the above-mentioned goals with a device for reprocessing composite plastic parts, for example molded parts produced from curable casting compositions, such as sanitary basins, comprising at least one filler and at least one polymer, in particular polymethyl methacrylate, in particular with a method according to one of claims 1 to 14, comprising a comminution device for comminuting the composite plastic parts to form a feed material, a feed device for feeding the feed material into a fluidized bed reactor with at least one fluidized material, the fluidized bed reactor for depolymerizing at least one polymer of the feed material by means of pyrolysis to form a pyrolysis product, in particular comprising methyl methacrylate, and separating the filler from the pyrolysis product, wherein the separated filler and the fluidized material are substantially similar in terms of material and/or size distribution, in particular the same, and a discharge device for discharging a fluid flow comprising the pyrolysis product from the fluidized bed reactor and for discharging at least part of the separated filler together with at least part of the fluidized material from the fluidized bed reactor.

One of the advantages of this is that there is no need for time-consuming separation of the discharged solids into filler and fluidized material. The discharged solids can be fed back into the fluidized bed reactor and/or collected to be used for the production of new composite plastic parts. This means that a more time and cost-efficient process can be provided. Furthermore, a separating device can be dispensed with as part of the device, which means that a simplified structure of the device can be provided.

The fluidized material can be whirled up from below through a porous plate in the fluidized bed reactor to form a fluidized bed. A fluidizing gas can be fed into the fluidized bed reactor. The feed material can be conveyed directly into the hot fluidized bed, whereupon the depolymerization of the at least one polymer into the pyrolysis product can take place. The reaction time can be in the range of a few seconds. As a result of the decomposition of the at least one polymer and the turbulence, the filler is separated from the pyrolysis product.

The term “substantially similar in material” is to be understood in the broadest sense and refers, in particular in the claims, preferably in the description, to a percentage match of the material compositions, in particular defined by the chemical composition and/or the microstructural properties, based on the volume and/or mass fractions of at least 80%, preferably at least 90%, in particular at least 95%. In this context, the term “identical in material” means a percentage match of at least 99%.

The term “substantially similar in size distribution” is to be understood in the broadest sense and refers, in particular in the claims, preferably in the description, to a percentage match of particle sizes and/or particle size fractions in relation to the volume and/or mass fractions of at least 80%, preferably at least 90%, in particular at least 95%. In this context, the term “equal in size distribution” means a percentage match of at least 99%.

Further features, advantages and further embodiments of the invention are described below or are disclosed thereby.

According to a preferred embodiment of the invention, at least part of the feed material has a particle size of at least 1 μm, preferably at least 0.1 mm, in particular at least 1 mm, and a maximum of 10 mm, preferably a maximum of 7 mm, in particular a maximum of 5 mm, in particular wherein the feed material is fed to the fluidized bed reactor by means of at least one screw conveyor. This enables particularly rapid depolymerization of the feed material, which can increase the efficiency of the process. A screw conveyor can be used to provide a process with a continuous supply of feed material with high dosing accuracy.

According to another preferred embodiment of the invention, at least part of the feed material has a particle size of between 50 mm and 100 mm, in particular wherein the feed material is fed to the fluidized bed reactor via at least one double flap sluice. One of the advantages of this is that it reduces the amount of work involved in shredding the composite plastic parts. A double flap sluice enables large quantities of feed material to be fed in quickly and easily.

According to another preferred embodiment of the invention, an operating temperature of the fluidized bed reactor is between 400° C. and 650° C., preferably between 425° C. and 500° C., in particular 450° C. One advantage of this is that most polymers can be depolymerized in these temperature ranges. An operating temperature of 450° C. is particularly suitable for depolymerizing polymethyl methacrylate by pyrolysis.

According to another preferred embodiment of the invention, the fluidized bed reactor is heated with the aid of at least one radiant heating tube. Radiant heating tubes enable the provision of a homogeneous temperature distribution within the fluidized bed reactor, which improves the reaction conditions and the efficiency of the pyrolysis process.

According to another preferred embodiment of the invention, the at least one radiant heating tube is operated at least partially with at least one excess gas from the pyrolysis, in particular wherein at least one of the at least one excess gas is separated from the fluid flow. This provides a particularly resource-efficient process.

According to another preferred embodiment of the invention, a fluidizing gas, in particular an inert gas such as nitrogen, is fed to the fluidized bed reactor at a pressure of between 140 mbar and 180 mbar, preferably 160 mbar, to generate a fluidized flow. In this way, an eddy current can be generated for homogeneous mixing and uniform temperature distribution in the fluidized bed reactor. The even temperature distribution prevents the formation of temperature concentrations that could lead to decomposition of the reaction material. The use of inert gases, such as nitrogen, reduces the likelihood of undesirable chemical reactions.

According to another preferred embodiment of the invention, the filler and the fluidized material comprise at least partially, in particular completely, inorganic material, for example quartz sand. Inorganic materials such as quartz sand have a high melting temperature and therefore exhibit high thermal stability. This allows the fluidized bed reactor to be operated at high temperatures without the filler melting or decomposing. This would lead to impurities in the pyrolysis product, which would have to be separated at great expense.

According to another preferred embodiment of the invention, the fluid flow is discharged from the fluidized bed reactor at a pressure of between 40 mbar and 60 mbar, preferably 50 mbar. This allows favorable reaction conditions to be provided for the pyrolysis process with a suitable pressure in the fluidized bed reactor.

According to another preferred embodiment of the invention, the fluid flow is fed to at least one centrifugal separator, in particular an aerocyclone, for separating solid particles. This reduces the wear on the device caused by solid particles. An aerocyclone is particularly suitable for the continuous separation of solid particles, which increases the purity of the discharged fluid flow.

According to another preferred embodiment of the invention, the fluid flow, in particular after being fed to the at least one centrifugal separator, is fed to at least one, preferably three, gas scrubbers. The advantage of this is that gaseous impurities and particles can be removed from the fluid flow. Soiling of the device, for example due to deposits, can thus be avoided.

According to another preferred embodiment of the invention, the fluid flow, in particular after being fed to the at least one gas scrubber, is fed to at least one electrostatic precipitator. One of the advantages achieved in this way is that particularly fine particles and aerosols, which cannot be completely removed by the at least one gas scrubber, can be separated. Soiling of the device, for example due to deposits, can thus be avoided.

According to another preferred embodiment of the invention, the pyrolysis product is separated from the fluid flow by condensation as pyrolysis oil. The separated pyrolysis product, which essentially contains monomeric reaction products, can thus be recycled. For example, new molded parts, such as sanitary basins, can be produced from hardenable casting compounds. It is conceivable that the pyrolysis oil is purified in order to increase the purity of the essentially monomeric reaction products obtained from the depolymerization of the at least one polymer.

According to another preferred embodiment of the invention, the fluid flow is conducted in a circuit, wherein the fluid flow is fed back to the fluidized bed reactor after separation of the pyrolysis product. One of the advantages of this is that it improves the material and energy efficiency of the process. In addition, the recirculation of the fluid flow helps to reduce waste products that have to be disposed of at great expense.

Further important features and advantages of the invention are apparent from the sub-claims, from the drawings and from the associated description of the figures with reference to the drawings.

It is understood that the features mentioned above and those to be explained below can be used not only in the combination indicated in each case, but also in other combinations or on their own, without departing from the scope of the present invention.

Preferred embodiments and embodiments of the present invention are shown in the drawings and are explained in more detail in the following description; wherein identical reference signs refer to identical or similar or functionally identical components or elements.

### SHOWING

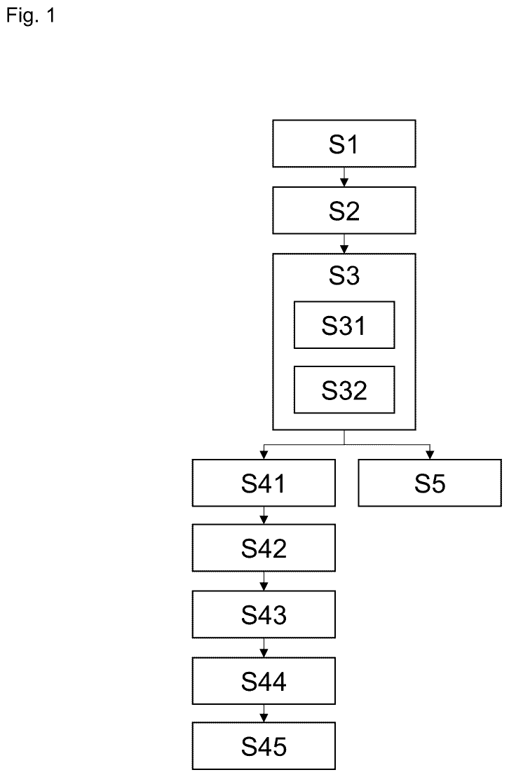

FIG. 1 Steps of a method according to an embodiment of the present invention, and

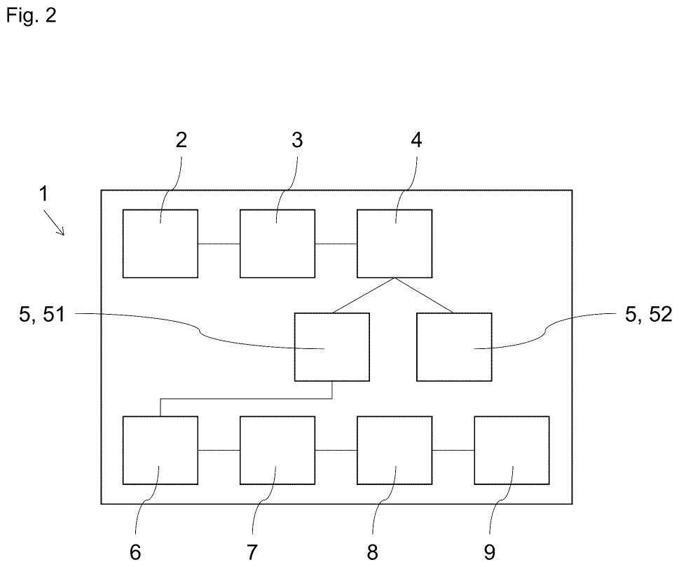

FIG. 2 Schematic representation of a device according to one embodiment of the present invention.

FIG. 1 shows steps of a method according to one embodiment of the present invention.

The process shown in FIG. 1 for reprocessing composite plastic parts is described below in relation to sanitary basins produced from curable casting compounds comprising a filler and polymers. The filler essentially comprises quartz sand and the polymers essentially comprise polymethyl methacrylate. It should be noted that the method described below can also be applied to other composite plastic parts comprising at least one filler and at least one polymer.

In a first step S1, the composite plastic parts are shredded into a feed material, which is then fed to a fluidized bed reactor with a fluidized material in step S2. The feed material is fed in by means of two screw conveyors and/or via a double flap sluice. The composite plastic parts are crushed to a particle size of at least 1 μm and a maximum of 10 mm for feeding using the screw conveyors. The composite plastic parts are shredded to a particle size of between 50 mm and 100 mm for feeding via the double flap feeder.

Step S3 comprises depolymerizing—step S31—the polymers by pyrolysis to a pyrolysis product, essentially comprising methyl methacrylate, and separating—step S32—the filler from the pyrolysis product. The swirl material and the separated filler are essentially similar in terms of material and size distribution. As part of this process, the fluidized material can be whirled up from below through a porous plate in the fluidized bed reactor to form a fluidized bed. Nitrogen is fed into the fluidized bed reactor as a fluidized gas at a pressure of 160 mbar. Several radiant heating tubes are used to heat the fluidized bed reactor to an operating temperature of 450° C. The feed material can be conveyed directly into the hot fluidized bed, whereupon the depolymerization of the polymers into the pyrolysis product can take place. As a result of the decomposition of the polymers and the turbulence, the filler is separated from the pyrolysis product.

The fluidized bed reactor can preferably have a diameter of 450 mm and a height of 900 mm. The height of the fluidized bed can preferably be 650 mm.

A fluid stream comprising the pyrolysis product with methyl methacrylate is then discharged from the fluidized bed reactor in step S41 at a pressure of 50 mbar and subjected to several treatment steps. In steps S42 and S43, the fluid flow is first fed to an aerocyclone to separate solid particles and then to three gas scrubbers. Subsequently, in step S44, the fluid flow is fed to an electrostatic precipitator in order to separate particularly fine particles and aerosols that cannot be completely removed by the three gas scrubbers in particular. In step S45, the pyrolysis product comprising methyl methacrylate is separated from the fluid stream by condensation as pyrolysis oil. The pyrolysis oil can then be further purified to obtain methyl methacrylate of high purity. The methyl methacrylate obtained can be used to produce new composite plastic parts.

In step S5, a portion of the separated filler is discharged from the fluidized bed reactor together with a portion of the fluidized bed material. The fluidized bed reactor can have an overflow for this purpose. The resulting quartz sand can also be used to produce new composite plastic parts.

FIG. 2 shows a schematic representation of a device according to one embodiment of the present invention.

The device 1 shown for recycling composite plastic parts, for example molded parts made from curable casting compounds such as sanitary basins, comprising at least one filler and at least one polymer, in particular polymethyl methacrylate, in particular using the method according to the embodiment of the present invention shown in FIG. 1, has a comminution device 2 for comminuting the composite plastic parts to form a feed material and a feed device 3 for feeding the feed material into a fluidized bed reactor 4 with at least one fluidized material.

The device 1 comprises the fluidized bed reactor 4 for depolymerizing the at least one polymer of the feed material by pyrolysis to a pyrolysis product, in particular comprising methyl methacrylate, and for separating the filler from the pyrolysis product. The separated filler and the fluidized material are essentially similar in terms of material and/or size distribution, in particular the same.

A discharge device 5 with two discharge units 51, 52 is connected to the fluidized bed reactor 4. A discharge unit 51 is designed to discharge a fluid flow, comprising the pyrolysis product, from the fluidized bed reactor 4. The other discharge unit 52 is designed to discharge at least a portion of the separated filler together with at least a portion of the fluidized bed material from the fluidized bed reactor 4.

Furthermore, the device 1 comprises a centrifugal separator 6 designed as an aerocyclone, three gas scrubbers 7, an electrostatic precipitator 8 and a condensing device 9, which is designed to separate the pyrolysis product from the fluid flow by condensation as pyrolysis oil.

In summary, at least one embodiment of the present invention may have at least one of the following features and/or may provide at least one of the following advantages:
More time and cost efficient process.
More energy-efficient process.
Reduction of the shredding effort.
Quick and easy feeding of the feed material.
Reduction of the probability of undesirable chemical reactions.
Reduction of wear and soiling.

Although the present invention has been described with reference to preferred embodiments, it is not limited thereto, but can be modified in a variety of ways.

### LIST OF REFERENCE SYMBOLS

1 Device
2 Comminution device
3 Feed device
4 Fluidized bed reactor
5 Discharge device
6 Centrifugal separator
7 Gas scrubber
8 Electrostatic precipitator
9 Condensing device
51, 52 Discharge units
S1-S5 Steps of a procedure
S31-S32 Steps of a procedure
S41-S45 Steps of a procedure

1. A method for recycling composite plastic parts, for example molded parts produced from curable casting compounds, such as sanitary basins, comprising at least one filler and at least one polymer, in particular polymethyl methacrylate, comprising the steps:
comminuting at least one composite plastic part into a feed material,
feeding the feed material into a fluidized bed reactor with at least one fluidized bed material,
depolymerizing the at least one polymer of the feed material by pyrolysis to a pyrolysis product, in particular comprising methyl methacrylate,
separating the filler from the pyrolysis product, wherein the separated filler and the fluidized material are substantially similar, in particular identical, in terms of material and/or size distribution,
discharging a fluid stream comprising the pyrolysis product from the fluidized bed reactor, and
discharging at least a portion of the separated filler together with at least a portion of the fluidized bed material from the fluidized bed reactor.

2. The method according to claim 1, characterized in that at least part of the feed material has a particle size of at least 1 μm, preferably at least 0.1 mm, in particular at least 1 mm, and at most 10 mm, preferably at most 7 mm, in particular at most 5 mm, in particular wherein the feed material is fed to the fluidized bed reactor by means of at least one screw conveyor.

3. The method according to claim 1, characterized in that at least a part of the feed material has a particle size between 50 mm and 100 mm, in particular wherein the feed material is fed to the fluidized bed reactor via at least one double flap sluice.

4. The method according to claim 1, characterized in that an operating temperature of the fluidized bed reactor is between 400° C. and 650° C., preferably between 425° C. and 500° C., in particular 450° C.

5. The method according to claim 1, characterized in that the fluidized bed reactor is heated by means of at least one radiant heating tube.

6. The method according to claim 5, characterized in that the at least one radiant heating tube is operated at least partially with at least one excess gas from the pyrolysis, in particular wherein at least one of the at least one excess gas is separated from the fluid flow.

7. The method according to claim 1, characterized in that a fluidized bed gas, in particular an inert gas, such as nitrogen, at a pressure of between 140 mbar and 180 mbar, preferably 160 mbar, is fed to the fluidized bed reactor to generate a fluidized flow.

8. The method according to claim 1, characterized in that the filler and the fluidized material comprise at least partially, in particular completely, inorganic material, for example quartz sand.

9. The method according to claim 1, characterized in that the fluid stream is discharged from the fluidized bed reactor at a pressure of between 40 mbar and 60 mbar, preferably of 50 mbar.

10. The method according to claim 1, characterized in that the fluid stream is fed to at least one centrifugal separator, in particular an aerocyclone, for separating solid particles.

11. The method according to claim 1, characterized in that the fluid stream, in particular after being fed to the at least one centrifugal separator, is fed to at least one, preferably three, gas scrubbers.

12. The method according to claim 1, characterized in that the fluid stream, in particular after being fed to the at least one gas scrubber, is fed to at least one electrostatic precipitator.

13. The method according to claim 1, characterized in that the pyrolysis product is separated from the fluid stream by condensation as pyrolysis oil.

14. The method according to claim 1, characterized in that the fluid flow is circulated, wherein the fluid flow is fed back to the fluidized bed reactor after separation of the pyrolysis product.

15. A device for recycling composite plastic parts, preferably molded parts produced from curable casting compounds, such as sanitary basins, comprising at least one filler and at least one polymer, in particular polymethyl methacrylate, in particular with a method according to claim 1, comprising
a comminution device for comminuting the composite plastic parts into a feed material,
a feed device for feeding the feed material into a fluidized bed reactor with at least one fluidized material,
a fluidized bed reactor for depolymerizing the at least one polymer of the feed material by means of pyrolysis to form a pyrolysis product, in particular comprising methyl methacrylate, and separating the filler from the pyrolysis product, wherein the separated filler and the fluidized material is substantially similar, in particular identical, in material and/or size distribution, and
a discharge device for discharging a fluid flow comprising the pyrolysis product from the fluidized bed reactor and for discharging at least a portion of the separated filler together with at least a portion of the fluidized material from the fluidized bed reactor.
